(19) United States
(12) Patent Application Publication
(10) Pub. No.: US 2021/0358636 A1
(43) Pub. Date: Nov. 18, 2021

(54) RAPID IDENTIFICATION OF OPTIMIZED COMBINATIONS OF INPUT PARAMETERS FOR A COMPLEX SYSTEM

(71) Applicant: THE REGENTS OF THE UNIVERSITY OF CALIFORNIA, Oakland, CA (US)

(72) Inventors: Chih-Ming Ho, Los Angeles, CA (US); Xianting Ding, Los Angeles, CA (US); Ieong Wong, Los Angeles, CA (US)

(21) Appl. No.: 17/390,505

(22) Filed: Jul. 30, 2021

Related U.S. Application Data
(62) Division of application No. 14/761,918, filed on Jul. 17, 2015.
(60) Provisional application No. 61/753,842, filed on Jan. 17, 2013.

(51) Int. Cl. G16H 50/50; G06F 17/11; G16B 5/00; G16C 20/70; G06F 30/20

(52) U.S. Cl. CPC G16H 50/50; G06F 17/11; G16H 10/20; G16C 20/70; G06F 30/20; G16B 5/00

(57) ABSTRACT

Multiple tests of a complex system are conducted by applying varying combinations of input parameters from a pool of input parameters. Results of the tests are fitted into a model of the complex system by using multi-dimensional fitting. Using the model of the complex system, identification is made of at least one optimized combination of input parameters to yield a desired response of the complex system.

### CROSS-REFERENCE TO RELATED APPLICATION

This application is a divisional of U.S. patent application Ser. No. 14/761,918, filed Jul. 17, 2015, which application is a national phase application under 35 USC 371 of PCT/US2014/012111 filed Jan. 17, 2014, which application claims the benefit of U.S. Provisional Application No. 61/753,842 filed on Jan. 17, 2013, the disclosures of which are incorporated herein by reference in their entirety.

### STATEMENT REGARDING FEDERALLY SPONSORED RESEARCH OR DEVELOPMENT

This invention was made with Government support under Grant No. 0751621, awarded by the National Science Foundation. The Government has certain rights in this invention.

### FIELD OF THE INVENTION

This disclosure generally relates to the identification of optimized input parameters for a complex system and, more particularly, to the identification of optimized combinations of input parameters for the complex system.

### BACKGROUND

Behaviors of complex systems, such as cells, animals, humans, and other biological, chemical, and physical systems, are often regulated by a set of internal and external control parameters. For example, a cancer cell can proliferate abnormally as a result of malfunction at multiple signaling pathways. In order to control such complex systems, combinations of control parameters are often desirable.

Specifically, taking the case of human immunodeficiency virus (HIV) as an example, the death rate of HIV patients kept increasing until drug combinations were applied in 1995. The death rate was reduced by about ⅔ in 2 years and stayed low afterwards. While a drug combination can be effective, developing optimized drug combinations for clinical trials can be extremely challenging. One of the reasons is that a drug combination being effective in vitro does not always indicate that the same drug-dosage combination would be effective in vivo. Traditionally, when a drug combination is successfully validated in vitro, the combination is applied in vivo, either by keeping the same dosage ratios or by adjusting the drug administration to achieve the same blood drug levels as attained in vitro. This approach can suffer from absorption, distribution, metabolism, and excretion (ADME) issues. ADME describes the disposition of a pharmaceutical compound within an organism, and the four characteristics of ADME can influence the drug levels, kinetics, and, therefore, efficacy of a drug combination. The discontinuity from cell line to animal as a result of ADME poses a major barrier to efficiently identifying optimized drug combinations for clinical trials.

It is against this background that a need arose to develop the combinatorial optimization technique described herein.

### SUMMARY

In one embodiment, a method of combinatorial optimization includes: (1) conducting multiple tests of a complex system by applying varying combinations of input parameters from a pool of input parameters; (2) fitting results of the tests into a model of the complex system by using multi-dimensional fitting; and (3) using the model of the complex system, identifying at least one optimized combination of input parameters to yield a desired response of the complex system.

In another embodiment, a method of combinatorial drug optimization includes: (1) conducting multiple in vivo or in vitro tests by applying varying combinations of drug dosages from a pool of drugs; (2) fitting results of the tests into a multi-dimensional response surface of drug efficacy; and (3) using the response surface, identifying at least one optimized combination of drug dosages to yield a desired drug efficacy.

In a further embodiment, a method of combinatorial optimization includes: (1) providing a model of a complex system, the model representing a response of the complex system as a low order function of N input parameters; and (2) using the model of the complex system, identifying multiple optimized sub-combinations of the N input parameters that yield desired responses of the complex system.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

For a better understanding of the nature and objects of some embodiments of this disclosure, reference should be made to the following detailed description taken in conjunction with the accompanying drawings.

FIG. 1 and FIG. 2 show examples of modeled herpes simplex virus 1 (HSV-1) response surfaces to drug combinations superimposed on experimental data, according to an embodiment of this disclosure.

FIG. 3 and FIG. 4 show examples of modeled lung cancer response surfaces to drug combinations superimposed on experimental data, according to an embodiment of this disclosure.

FIG. 5 shows an example of identifying an optimized dosage with 3 tests for one input parameter, according to an embodiment of this disclosure.

FIG. 6 shows a processing unit implemented in accordance with an embodiment of this disclosure.

### DETAILED DESCRIPTION

### Overview

Embodiments of this disclosure are directed to identifying optimized combinations of input parameters for a complex system. Advantageously, embodiments of this disclosure circumvent several major technology roadblocks encountered in optimizing complex systems, such as related to labor, cost, risk, reliability, efficacies, side effects, and toxicities. The goal of optimization of some embodiments of this disclosure can be any one or any combination of reducing labor, reducing cost, reducing risk, increasing reliability, increasing efficacies, reducing side effects, and reducing toxicities, among others. In some embodiments, a specific example of treating diseases of a biological system with optimized drug combinations (or combinatorial drugs) and respective dosages is used to illustrate certain aspects of this disclosure. A biological system can include, for example, an individual cell, a collection of cells such as a cell culture or a cell line, an organ, a tissue, or a multi-cellular organism such as an animal, an individual human patient, or a group of human patients. A biological system can also include, for example, a multi-tissue system such as the nervous system, immune system, or cardio-vascular system.

More generally, embodiments of this disclosure can optimize wide varieties of other complex systems by applying pharmaceutical, chemical, nutritional, physical, or other types of stimulations or control parameters. Applications of embodiments of this disclosure include, for example, optimization of drug combinations, vaccine or vaccine combinations, chemical synthesis, combinatorial chemistry, drug screening, treatment therapy, cosmetics, fragrances, and tissue engineering, as well as other scenarios where a group of optimized input parameters is of interest. For example, other embodiments can be used for 1) optimizing design of a large molecule (e.g., drug molecule or protein and aptamer folding), 2) optimizing the docking of a molecule to another molecule for biomarker sensing, 3) optimizing the manufacturing of materials (e.g., from chemical vapor deposition (CVD) or other chemical system), 4) optimizing alloy properties (e.g., high temperature super conductors), 5) optimizing a diet or a nutritional regimen to attain desired health benefits, 6) optimizing ingredients and respective amounts in the design of cosmetics and fragrances, 7) optimizing an engineering or a computer system (e.g., an energy harvesting system, a computer network, or the Internet), and 8) optimizing a financial market.

Input parameters can be pharmaceutical (e.g., drugs), biological (e.g., cytokines and kinase inhibitors), chemical (e.g., chemical compounds), electrical (e.g., electrical current or pulse), and physical (e.g., thermal energy and pressure or shear force), among others. Optimization can include complete optimization in some embodiments, but also can include substantially complete or partial optimization in other embodiments.

Embodiments of this disclosure provide a number of benefits. For example, current drug discovery relies greatly on high throughput screening (HTS), which applies brute force screening of millions of chemical, genetic, or pharmacological tests. Such technique has high cost, is labor-intensive, and generates a high amount of waste and low information density data. Besides the intensive labor and cost involved in current in vitro drug screening, another issue with current drug screening lies in the transfer of knowledge between in vitro and in vivo studies. A problem of in vitro experimental studies is that in vitro results sometimes are not able to be extrapolated to in vivo systems and can lead to erroneous conclusions. There are also instances where metabolic enzymes in the body perform very differently between in vitro and in vivo, and these differences can tremendously alter drug activity and potentially increase the risk of underestimation of toxicity. Some embodiments of this disclosure can bypass the above-noted disadvantages of current drug screening. Specifically, some embodiments can effectively replace the intensive labor and cost procedures of in vitro drug screening with a minimal or reduced amount of in vivo studies, thereby greatly enhancing the reliability and applicability of experimental results.

Traditionally, knowledge from cell line studies is not readily transferrable to animal model or clinical studies. This barrier is referred to as roadblocks in biological research, and poses a challenge to successfully identifying effective drug combinations. One of the benefits of some embodiments of this disclosure is that the technique can bypass in vitro studies and directly identify optimized drug-dosage combinations in vivo, overcoming the challenge of discontinuity.

Animal testing is a useful tool during drug development, such as to test drug efficacy, to identify potential side effects, and to identify safe dosage in humans. However, animal testing can be highly labor and cost-intensive. One of the benefits of some embodiments of this disclosure is that the technique can reduce or minimize the amount of animal testing.

Current efforts in identifying optimized drug combinations have largely focused on 2 or 3 drugs with a few dosages on a trial-by-error basis. When the number of drugs and dosages increase, current combinatorial drug development becomes prohibitive. One of the benefits of some embodiments of this disclosure is that the technique provides a systematic approach to identify at least a subset, or all, optimized drug-dosage combinations from a pool of a large number of drugs, while maintaining the number of in vivo tests to a manageable number.

### Optimized Combinations of Input Parameters for a Complex System

Stimulations can be applied to direct a complex system toward a desired state, such as applying drugs to treat a patient. The types and the amplitudes (e.g., dosages) of applying these stimulations are part of the input parameters that can affect the efficiency in bringing the system toward the desired state. However, N types of different drugs with M dosages for each drug will result in M^{N} possible drug-dosage combinations. To identify an optimized or even near optimized combination by multiple tests on all possible combinations is prohibitive in practice. For example, it is not practical to perform all the possible drug-dosage combinations in animal and clinical tests for finding an effective drug-dosage combination as the number of drugs and dosages increase.

Embodiments of this disclosure provide a technique that allows a rapid search for optimized combinations of input parameters to guide multi-dimensional (or multivariate) engineering, medicine, financial, and industrial problems, as well as controlling other complex systems with multiple input parameters toward their desired states. The technique is comprised of a multi-dimensional complex system whose state is affected by input parameters along respective dimensions of a multi-dimensional parameter space. In some embodiments, the technique can efficiently operate on a large pool of input parameters (e.g., a drug library), where the input parameters can involve complex interactions both among the parameters and with the complex system. A search technique can be used to identify at least a subset, or all, optimized combinations or sub-combinations of input parameters that produce desired states of the complex system. Taking the case of combinational drugs, for example, a large number of drugs can be evaluated to rapidly identify optimized combinations, ratios, and dosages of drugs. A parameter space sampling technique (e.g., an experimental design methodology) can guide the selection of a minimal or reduced number of tests to expose salient features of the complex system being evaluated, and to reveal a combination or sub-combination of input parameters of greater significance or impact in affecting a state of the complex system.

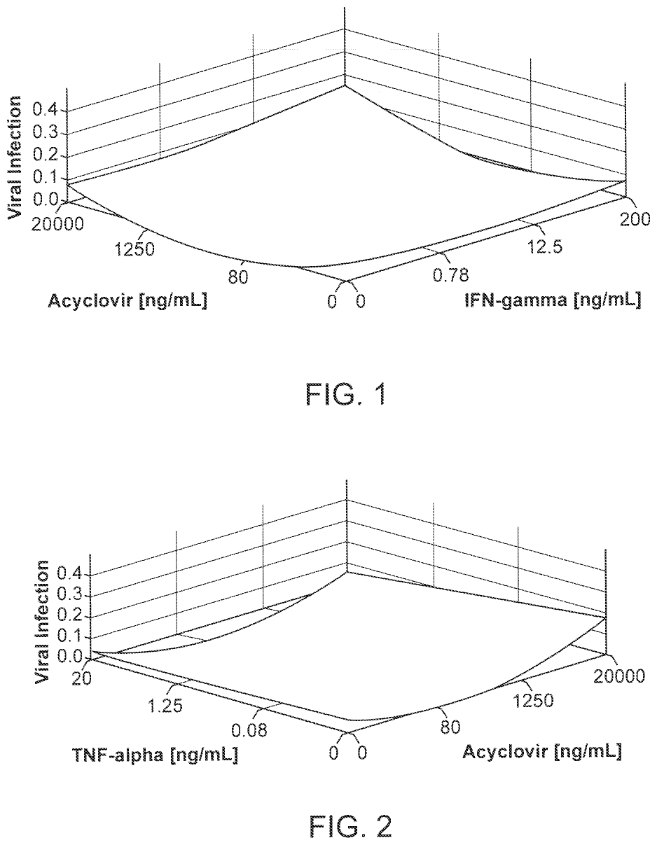

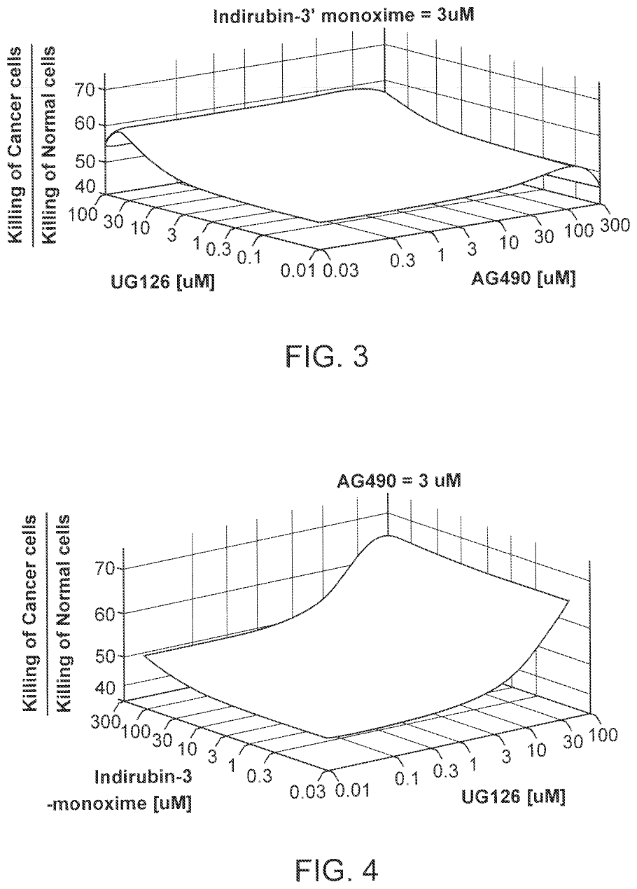

Embodiments of this disclosure are based on a surprising finding that a response of a complex system to multiple input parameters can be represented by a low order equation, such as a second order (or quadratic) equation, although a first order (or linear) equation as well as a third order (or cubic) equation are also contemplated as possible low order equations. Also, higher order equations are also contemplated for other embodiments. Taking the case of combinational drugs, for example, a drug efficacy E can be represented as a function of drug dosages as follows:

$$E = {E}_{0} + {\sum }_{i} {a}_{i} {C}_{i} + {\sum }_{i , j} {a}_{ij} {C}_{i} {C}_{j} + O ( {C}_{i} {C}_{j} {C}_{k} )$$

where Cᵢ is a dosage of an i^{th} drug from a pool of N total drugs, E₀ is a constant representing a baseline efficacy, a is a constant representing a single drug efficacy coefficient, aᵢⱼ is a constant representing a drug-drug interaction coefficient, and the summations run through N. If cubic and other higher order terms are omitted, then the drug efficacy E can be represented by a quadratic model as a function of the drug dosages Cᵢ. FIG. 1 and FIG. 2 show examples of modeled herpes simplex virus 1 (HSV-1) response surfaces to drug combinations superimposed on experimental data, demonstrating that the experimental data is smooth and can be represented by quadratic models. FIG. 3 and FIG. 4 show examples of modeled lung cancer response surfaces to drug combinations superimposed on experimental data, again demonstrating that the experimental data is smooth and can be represented by quadratic models. As noted above, other models, including ternary and higher order models or the use of linear regression model, are also contemplated. Also, although a specific example of combinational drugs is used, it should be noted that the above equation more generally can be used to represent a wide variety of other complex systems as a function of multiple input parameters.

For the case of N=1 (a pool of 1 drug), then:

E=E₀+a₁C₁+a₁₁C₁C₁

with a total of three constants, E₀, a₁, and a₁₁.

For the case of N=2 (a pool of 2 drugs), then:

E=E₀+a₁C₁+a₂C₂+a₁₂C₁C₂+a₁₁C₁C₁+a₂₂C₂C₂

with a total of six constants, E₀, a₁, a₂, a₁₂, a₁₁, and a₂₂.

More generally for N total drugs, a total number of constants m is 1+2N+(N(N−1))/2. If one drug dosage is kept constant in the study, the number of constants m can be further reduced to 1+2(N−1)+((N−1)(N−2))/2, for N>1. Table 1 below sets forth a total number of constants in a quadratic model of drug efficacy as a function of a total number drugs in a pool of drugs being evaluated.

TABLE 1

| | | Constants (m) (if one drug |
|---|---|---|
| Drugs (N) | Constants (m) | dosage is kept constant) |
| 1 | 3 | — |
| 2 | 6 | 3 |
| 3 | 10 | 6 |
| 4 | 15 | 10 |
| 5 | 21 | 15 |
| 6 | 28 | 21 |

By leveraging this surprising finding, a relatively small number of in vivo tests (e.g., animal tests) can be conducted to model an efficacy-dosage response surface, and this input/output model can be used to identify optimized drug-dosage combinations. In some embodiments, the in vivo tests can be conducted in parallel in a single in vivo study, thereby greatly enhancing the speed and lowering labor and costs compared with current drug screening.

Taking the case of the quadratic model of drug efficacy E, for example, different combinations of the drug dosages Cᵢ can be selected for respective in vivo tests as follows:

$${E}^{1} = {E}_{0} + {\sum }_{i} {a}_{i} {C}_{i}^{1} + {\sum }_{i , j} {a}_{ij} {C}_{i}^{1} {C}_{j}^{1}$$

$${E}^{2} = {E}_{0} + {\sum }_{i} {a}_{i} {C}_{i}^{2} + {\sum }_{i , j} {a}_{ij} {C}_{i}^{2} {C}_{j}^{2}$$

$$…$$

$${E}^{n} = {E}_{0} + {\sum }_{i} {a}_{i} {C}_{i}^{n} + {\sum }_{i , j} {a}_{ij} {C}_{i}^{n} {C}_{j}^{n}$$

where E^{k} is an efficacy observed or measured in a k^{th} test from a total of n tests, and Cᵢ^{k} is a dosage of an i^{th} drug applied in the k^{th} test. From the n tests, the m constants E₀, aᵢ, and aᵢⱼ can be derived, with n≥m, namely with the number of tests being the same as, or greater than, the number of constants in the quadratic model. In some embodiments, a minimal number of tests can be conducted, with n=m. If one drug dosage is kept constant in the study, the number of tests n can be further reduced to 1+2(N−1)+((N−1)(N−2))/2, for N>1.

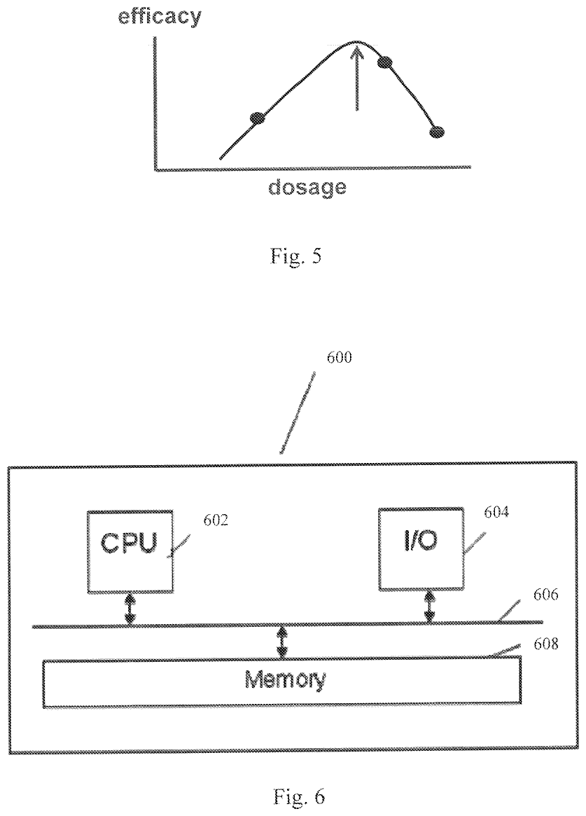

In some embodiments, an experimental design methodology can be used to guide the selection of drug dosages for respective in vivo tests. In connection with the experimental design methodology, possible dosages can be narrowed down into a few discrete levels. FIG. 5 shows an example of the design of tests to model an efficacy-dosage response surface. As shown in FIG. 5, the tests are designed such that at least one tested dosage lies on either side of a peak or maximum in the response surface in order to model the surface as a quadratic function.

Once tests are designed and conducted, experimental results of the tests (e.g., in terms of efficacies E^{k}) are then fitted into a model by using any suitable multi-dimensional fitting, such as regression analysis. Based on the fitting performance between the experimental results and the model, additional tests can be conducted to improve the accuracy of the model. Once the model with a desired accuracy is achieved, optimized combinations of input parameters of the system can be identified by using any suitable extrema locating technique, such as by locating global or local maxima in a response surface. FIG. 5 shows an example of identifying an optimized dosage of a single drug regimen with 3 tests.

Taking the case of the quadratic model of drug efficacy E, for example, optimized dosages can be identified once the constants E₀, and aᵢⱼ are derived through multi-dimensional fitting:

$${E}_{max} = {E}_{0} + {\sum }_{i} {a}_{i} {\hat{C}}_{i} + {\sum }_{i . j} {a}_{ij} {\hat{C}}_{i} {\hat{C}}_{j}$$

where {Ĉᵢ} is an optimized dosage of an i^{th} drug from the pool of N total drugs.

In the case of a relatively large pool of drugs being evaluated (e.g., N≥10, 100, or even 1,000 or more), optimized sub-combinations of drugs can be identified to facilitate subsequent clinical trials in human patients. For example, in the case of a pool of 6 total drugs, all optimized sub-combinations of 3 drugs from the pool of drugs can be identified, by setting dosages of 3 drugs in the pool to zero to effectively reduce a 6-dimensional system to a 3-dimensional system, and locating maxima with respect to the 3 remaining dimensions. In this example of the pool of 6 drugs, a total of 20 different optimized sub-combinations of 3 drugs can be identified. Also, still in the case of the pool of 6 drugs, all optimized sub-combinations of 4 drugs from the pool of drugs can be identified, by setting dosages of 2 drugs in the pool to zero to effectively reduce the 6-dimensional system to a 4-dimensional system, and locating maxima with respect to the 4 remaining dimensions. In this example of the pool of 6 drugs, a total of 15 different optimized sub-combinations of 4 drugs can be identified. Thus, by conducting as few as 28 in vivo tests for the pool of 6 drugs, 35 (=20+15) optimized sub-combinations of 3 and 4 drugs can be identified as candidates for clinical trials. In other embodiments, in vitro tests can be conducted to identify all optimized sub-combinations, and then a subset that is most suitable can be selected for animal tests. A similar procedure can be conducted in moving from animal tests to clinical trials.

Once a model with a desired accuracy is achieved for some embodiments, the significance of each input parameter and its synergistic effect with other input parameters can be identified. Non-significant input parameters that have little or no impact in affecting a state of a complex system can be dropped or omitted from an initial pool of input parameters, thereby effectively converting an initial multi-dimensional system to a refined system with a lower dimensionality. Taking the case of the quadratic model of drug efficacy E, for example, non-significant drugs can be identified as having low values of the constants aᵢ and aᵢⱼ, and can be dropped from an initial pool of drugs for subsequent evaluation.

### Processing Unit

FIG. 6 shows a processing unit 600 implemented in accordance with an embodiment of this disclosure. Depending on the specific application, the processing unit 600 can be implemented as, for example, a portable electronics device, a client computer, or a server computer. Referring to FIG. 6, the processing unit 600 includes a central processing unit (“CPU”) 602 that is connected to a bus 606. Input/Output (“I/O”) devices 604 are also connected to the bus 606, and can include a keyboard, mouse, display, and the like. An executable program, which includes a set of software modules for certain procedures described in the foregoing sections, is stored in a memory 608, which is also connected to the bus 606. The memory 608 can also store a user interface module to generate visual presentations.

An embodiment of this disclosure relates to a non-transitory computer-readable storage medium having computer code thereon for performing various computer-implemented operations. The term “computer-readable storage medium” is used herein to include any medium that is capable of storing or encoding a sequence of instructions or computer codes for performing the operations described herein. The media and computer code may be those specially designed and constructed for the purposes of this disclosure, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as floptical disks; and hardware devices that are specially configured to store and execute program code, such as application-specific integrated circuits (ASICs), programmable logic devices (PLDs), and ROM and RAM devices. Examples of computer code include machine code, such as produced by a compiler, and files containing higher-level code that are executed by a computer using an interpreter or a compiler. For example, an embodiment of the invention may be implemented using Java, C++, or other object-oriented programming language and development tools. Additional examples of computer code include encrypted code and compressed code. Moreover, an embodiment of the invention may be downloaded as a computer program product, which may be transferred from a remote computer (e.g., a server computer) to a requesting computer (e.g., a client computer or a different server computer) via a transmission channel. Another embodiment of the invention may be implemented in hardwired circuitry in place of, or in combination with, machine-executable software instructions.

As used herein, the singular terms “a,” “an,” and “the” include plural referents unless the context clearly dictates otherwise. Thus, for example, reference to an object can include multiple objects unless the context clearly dictates otherwise.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. For example, the terms can refer to less than or equal to ±5%, such as less than or equal to ±4%, less than or equal to ±3%, less than or equal to ±2%, less than or equal to ±1%, less than or equal to ±0.5%, less than or equal to ±0.1%, or less than or equal to ±0.05%.

While the invention has been described with reference to the specific embodiments thereof, it should be understood by those skilled in the art that various changes may be made and equivalents may be substituted without departing from the true spirit and scope of the invention as defined by the appended claims. In addition, many modifications may be made to adapt a particular situation, material, composition of matter, method, operation or operations, to the objective, spirit and scope of the invention. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the invention. Accordingly, unless specifically indicated herein, the order and grouping of the operations is not a limitation of the invention.

What is claimed is:

1. A method, comprising:
for a pool of drugs including N total drugs with N being 3 or greater, representing a multi-dimensional response surface of drug efficacy as a quadratic function of drug dosages with m coefficients, and m=1+2N+(N(N−1))/2 or one drug dosage from the pool of drugs is kept constant, and m=1+2(N−1)+((N−1)(N−2))/2;
based on m, designing n experimental tests of drugs on biological samples, with n≥m;
conducting the n tests by applying varying combinations of drug dosages from the pool of drugs;
fitting results of the n tests into the response surface of drug efficacy;
using the response surface of drug efficacy, identifying optimized sub-combinations of drugs from the pool of drugs;
selecting an optimized sub-combination of drugs to yield a desired drug efficacy from the optimized sub-combinations of drugs, the selected sub-combination of drugs having a number of drugs that is less than N; and
evaluating the selected sub-combination of drugs to identify an optimized combination of dosages of the selected sub-combination of drugs.

2. The method of claim 1, wherein N≥6.

3. The method of claim 1, wherein N≥10.

4. The method of claim 1, wherein fitting results is performed using multi-dimensional fitting.

5. The method of claim 1, wherein fitting the results of the tests includes deriving values of the m coefficients.

6. The method of claim 1, wherein identifying the at least one optimized sub-combination of drugs includes identifying at least one extremum in the response surface.

7. The method of claim 1, wherein the n tests are the result of a space interrogated based on a design of experiments.

8. The method of claim 7, wherein the design of experiments comprises orthogonal array composite design (OACD).

9. The method of claim 7, wherein the design of experiments combines fractional factorial and composite design.

10. The method of claim 1, wherein the biological samples of the experimental drug tests are cell lines.

11. The method of claim 1, wherein the biological samples of the experimental drug tests are animal models.

12. The method of claim 1, wherein the biological samples of the experimental drug tests are ex vivo cell cultures.

13. The method of claim 1, wherein the biological samples of the experimental drug tests are ex vivo tissues.

14. The method of claim 8, wherein the biological samples of the experimental drug tests are animal models.

15. The method of claim 8, wherein the biological samples of the experimental drug tests are ex vivo cell cultures.

16. The method of claim 8, wherein the biological samples of the experimental drug tests are ex vivo tissues.

17. The method of claim 9, wherein the biological samples of the experimental drug tests are animal models.

18. The method of claim 9, wherein the biological samples of the experimental drug tests are ex vivo cell cultures.

19. The method of claim 9, wherein the biological samples of the experimental drug tests are ex vivo tissues.
